(19) United States
(12) Patent Application Publication
(10) Pub. No.: US 2026/0097114 A1
(43) Pub. Date: Apr. 9, 2026

(54) Methods for the Rapid Manufacture of Conjugate Vaccines that Elicit Robust Immune Responses

(71) Applicant: Inventprise, Inc., Redmond, WA (US)

(72) Inventor: Subhash V. Kapre, Redmond, WA (US)

(73) Assignee: Inventprise, Inc., Redmond, WA (US)

(21) Appl. No.: 19/352,239

(22) Filed: Oct. 7, 2025

Related U.S. Application Data
(60) Provisional application No. 63/704,161, filed on Oct. 7, 2024.

(51) Int. Cl. A61K 39/215; A61K 9/00; A61K 39/00; A61K 39/02; A61K 39/385; A61K 39/39; A61P 31/14

(52) U.S. Cl. CPC A61K 39/215; A61K 9/0019; A61K 39/02; A61K 39/385; A61K 39/39; A61P 31/14; A61K 2039/54; A61K 2039/545; A61K 2039/55505; A61K 2039/55555; A61K 2039/55572; A61K 2039/55577; A61K 2039/6037; A61K 2039/70

(57) ABSTRACT

The disclosures of the invention are directed to the manufacture of effective, affordable vaccines that are globally accessible. Using a platform conjugation technology, highly immunogenic conjugate vaccines were produced that elicit broad cross-neutralization to variants of concern (VOC), manufactured cheaply compared to mRNA vaccines. Protein-protein conjugates and Toll-Like Receptor (TLR) agonist adjuvants were shown to enhance immunogenicity and induce broad cross-protection against VOC, a characteristic lacking in early mRNA COVID-19 vaccines. Murine nAb titers from Beta-only conjugates were equivalent between Beta, Delta, Omicron BA.1, BA.2, and BA.4/BA.5, which were circulating up to three years after the antigenic strain. Additionally, Beta-Delta bivalent conjugate vaccines readily prevented disease in hamster challenge, which demonstrates a vaccine with remarkably broad cross-protection and potential to protect for extended periods despite mutations, without requiring expensive boosters or antigen adaption. This vaccine can be produced in our highly automated, large-scale manufacturing facility enabling economical production of inexpensive, effective vaccines for high-need areas.

### REFERENCE TO RELATED APPLICATIONS

This application claims priority to U.S. Provisional Application No. 63/704,161, filed Oct. 7, 2024, the entirety of which is incorporated by reference.

### BACKGROUND

### 1. Field of the Invention

The invention is directed to manufacture of immunogenic compositions using mRNA sequences that encode antigens in established cell lines, collecting the antigens that are expressed and, preferably conjugating the antigen with other substances, in particular, toll-like receptor agonists. These highly immunogenic conjugate compositions and vaccines elicit broad cross-neutralization to variants of concern (VOC).

### 2. Description of the Background

The global impact of COVID-19 was dramatically mitigated by the rapid roll-out of effective vaccines that prevented death and serious illness from the SARS-CoV-2 virus. In their first year of use, COVID-19 vaccines were attributed with reducing COVID-19 deaths by 63% in 185 countries, a truly remarkable achievement; however, most of these prevented mortalities were in high-income countries because of vaccine inequity. Although mRNA vaccines can be generated swiftly, the breadth of protection and effectiveness offered by first generation mRNA vaccines waned rapidly, warranting frequent boosters to maintain adequate protection against disease. COVID-19 is now endemic and continually evolving, so broadly protective vaccines that confer durable protection against the continually evolving SARS-CoV-2 virus are still required, particularly in Low-and Middle-Income Countries (LMIC).

The SARS-CoV-2 spike protein harbors the Receptor Binding Domain (RBD) which interacts with the Angiotensin Converting Enzyme 2 (ACE2) receptor, located on the host cell's surface, to gain cellular entry upon infection. The RBD is the primary binding site of neutralizing antibodies and is the most appropriate antigen for vaccine candidate development. In its natural form the spike protein is metastable, existing in a prefusion state before binding the ACE2 receptor, then undergoing conformational transformation after binding. To ensure correct protein conformation for immunogenic recognition of the RBD and an appropriately targeted immune response, stabilized prefusion spike protein that cannot undergo transformational change is essential. Two proline stabilized trimerized spike protein (S-2P) expressed in stable CHO cell pools was selected as the primary antigen for its immunogenicity, yields, rapid production, stability profile, and affordability. Good Manufacturing Practice (GMP) stable CHO pools can be generated in as little as 8 weeks and exhibit comparable quality to clonal cells. By using CHO pools, new pathogens and immuno-evasive strains can be addressed quickly, accelerating the approval pipeline. Accordingly, revision to regulatory guidelines allowing vaccine antigens derived from pooled cell populations in early clinical phases is warranted.

Accordingly, there is a need to reduce the overall cost of producing vaccines and also, to rapid develop vaccine against rapidly emerging variants.

### SUMMARY OF THE INVENTION

The present invention overcomes the problems and disadvantages associated with current strategies and designs and provides new method for the rapid manufacture of immunogenic compositions and vaccines and in a cost-effective manner.

One embodiment of the invention is directed to methods for the manufacture of an immunogenic composition comprising: providing an RNA sequence that encodes an antigenic determinant; expressing an antigen from the RNA sequence in a cell line; and collecting the antigen expressed. Preferably the antigenic determinant is an immunologically active antigen such as an infectious agent of a species, subspecies or serotypes of Staphylococcus, Streptococcus, Salmonella, Pneumococcus, Campylobacter, Pseudomonas, Neisseria, Mycobacteria, Corynebacteria, Enterobacter, Vibrio, Bordetella, Clostridium, Yersinia, Listeria, Treponema, or another infectious microorganism. Preferably the method further comprising combining the antigen to a biological substance to form an immunogenic composition such as a carrier protein. Preferably the carrier protein is selected from the group consisting of tetanus toxoid, diphtheria toxoid, CRM197, tetanus toxoid fragments (TTHc), N. meningitidis protein PorB, RSV virus proteins, B. Pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof. Preferably the composition further comprises an adjuvant such as, for example, aluminum salt, calcium phosphate, a liposome of monophosphoryl lipid A (MPLA), saponin QS-21, TLR ligands, and/or a potent TLR4/7/8/9 agonists. Preferably the carrier protein is conjugated to the antigen. Also preferably, the carrier protein is a toll-like receptor agonist.

Another and related embodiment of the invention is an antigen manufactured by the methods disclosed herein.

Another and related embodiment of the invention comprises methods of administering the antigen manufactured by the methods disclosed herein to treat or prevent an infection.

Other embodiments and advantages of the invention are set forth in part in the description, which follows, and in part, may be obvious from this description, or may be learned from the practice of the invention.

### DESCRIPTION OF THE DRAWINGS

FIG. 1A. Induction time course of S-2P protein expression up to 11 days. S-2P Beta SDS PAGE gel.

FIG. 1B. Induction time course of S-2P protein expression up to 11 days. Representation of fold change in expression over time of induction.

FIG. 1C. Induction time course of S-2P protein expression up to 11 days. S-2P Delta SDS-PAGE gel.

FIG. 1D. Induction time course of S-2P protein expression up to 11 days. Representation of fold change in expression over time of induction.

FIG. 2A. Representative S-2P Beta chromatography data as shown in AKTA Purification Chromatogram.

FIG. 2B. Representative S-2P Beta chromatography data as shown in SDS-PAGE.

FIG. 3A. Dose, Conjugation and 3M-052 improve immune responses in mice: Young female BALB/c mice (Protein+Al(OH)3 n=7, Conjugate+Al(OH)3 n=7, Conjugate+Al(OH)3+3M-052 n=8) were inoculated intramuscularly with two doses of each vaccine candidate or placebo (PBS) 14 days apart. Mouse sera collected 2 weeks post-boost (Day 28), was tested for specific binding IgG to Wuhan S-2P. Data is displayed as Geometric Mean±95% CI. Two-tailed Mann-Whitney tests were performed to determine statistical significance.

FIG. 3B. Dose, Conjugation and 3M-052 improve immune responses in mice: Young female BALB/c mice (Protein+Al(OH)3 n=7, Conjugate+Al(OH)3 n=7, Conjugate+Al(OH)3+3M-052 n=8) were inoculated intramuscularly with two doses of each vaccine candidate or placebo (PBS) 14 days apart. Mouse sera collected 2 weeks post-boost (Day 28), was tested for specific binding to neutralizing antibodies. Data is displayed as Geometric Mean±95% CI. Two-tailed Mann-Whitney tests were performed to determine statistical significance.

FIG. 4A. Binding IgG to Beta and Delta SARS-CoV-2 S-2P. Binding IgG endpoint titers were tested in mouse sera collected at day 35 (Beta). Endpoint titers are defined as the reciprocal of the highest detectable dilution above the cutoff value, where the cutoff is the background+3 SD of background. For day 35 analysis all bivalent groups and all groups with CpG 1018 contained 7 animals/group, while Beta-only and Delta-Only with only Al(OH)3 contained 6 animals/group. Day 49 data contains 4 animals/group for all bivalent groups and all CpG 1018 groups, while the Beta-only and Delta-Only with only Al(OH)3 had 3 animals/group. Data shown as Geometric Mean±95% CI. One-way ANOVA with Dunn's multiple comparison to placebo was performed to determine statistical significance.

FIG. 4B. Binding IgG to Beta and Delta SARS-CoV-2 S-2P. Binding IgG endpoint titers were tested in mouse sera collected at day 35 (Delta). Endpoint titers are defined as the reciprocal of the highest detectable dilution above the cutoff value, where the cutoff is the background+3 SD of background. For day 35 analysis all bivalent groups and all groups with CpG 1018 contained 7 animals/group, while Beta-only and Delta-Only with only Al(OH)3 contained 6 animals/group. Day 49 data contains 4 animals/group for all bivalent groups and all CpG 1018 groups, while the Beta-only and Delta-Only with only Al(OH)3 had 3 animals/group. Data shown as Geometric Mean±95% CI. One-way ANOVA with Dunn's multiple comparison to placebo was performed to determine statistical significance.

FIG. 4C. Binding IgG to Beta and Delta SARS-CoV-2 S-2P. Binding IgG endpoint titers were tested in mouse sera collected at day 49 (Beta). Endpoint titers are defined as the reciprocal of the highest detectable dilution above the cutoff value, where the cutoff is the background+3 SD of background. For day 35 analysis all bivalent groups and all groups with CpG 1018 contained 7 animals/group, while Beta-only and Delta-Only with only Al(OH)3 contained 6 animals/group. Day 49 data contains 4 animals/group for all bivalent groups and all CpG 1018 groups, while the Beta-only and Delta-Only with only Al(OH)3 had 3 animals/group. Data shown as Geometric Mean±95% CI. One-way ANOVA with Dunn's multiple comparison to placebo was performed to determine statistical significance.

FIG. 4D. Binding IgG to Beta and Delta SARS-CoV-2 S-2P. Binding IgG endpoint titers were tested in mouse sera collected at day 49 (Delta). Endpoint titers are defined as the reciprocal of the highest detectable dilution above the cutoff value, where the cutoff is the background+3 SD of background. For day 35 analysis all bivalent groups and all groups with CpG 1018 contained 7 animals/group, while Beta-only and Delta-Only with only Al(OH)3 contained 6 animals/group. Day 49 data contains 4 animals/group for all bivalent groups and all CpG 1018 groups, while the Beta-only and Delta-Only with only Al(OH)3 had 3 animals/group. Data shown as Geometric Mean±95% CI. One-way ANOVA with Dunn's multiple comparison to placebo was performed to determine statistical significance.

FIG. 5A. Neutralizing Antibody Titers to Variants of Concern in Day 35 Mouse Sera Neutralizing antibody titers in mouse sera from day 35. Data is presented as GMT±95% CI. For day 35 analysis all bivalent groups and all groups with CpG 1018 contained 7 animals/group, while Beta-only and Delta-Only with only Al(OH)3 contained 6 animals/group. Day 49 data is obtained from 4 animals/group for bivalent and CpG 1018 groups, 3 animals/group for Beta-only and Delta-Only with only Al(OH)3. To determine if sera from the same group neutralized the SARS-CoV-2 variants differently, One-Way ANOVA with Dunn's Multiple Comparison was performed on results of each vaccine group.

FIG. 5B. Neutralizing Antibody Titers to Variants of Concern in Day 49 Mouse Sera Neutralizing antibody titers in mouse sera from day day 49. Data is presented as GMT±95% CI. For day 35 analysis all bivalent groups and all groups with CpG 1018 contained 7 animals/group, while Beta-only and Delta-Only with only Al(OH)3 contained 6 animals/group. Day 49 data is obtained from 4 animals/group for bivalent and CpG 1018 groups, 3 animals/group for Beta-only and Delta-Only with only Al(OH)3. To determine if sera from the same group neutralized the SARS-CoV-2 variants differently, One-Way ANOVA with Dunn's Multiple Comparison was performed on results of each vaccine group.

FIG. 6. Timeline of SARS-CoV-2 Variant Cases Worldwide case numbers of SARS-CoV-2 variants over time were obtained from https://ourworldindata. org/grapher/covid-variants-area sourced from GISAID, via CoVariants. org and plotted to assess theoretical longevity of conjugate vaccine candidates, based on equal murine nAb serological titers to VOC tested via pseudovirus neutralization.

FIG. 7A. Bivalent SARS-CoV-2 vaccine candidate protected hamsters when challenged with Beta and Delta—Post-challenge body weight are shown. Assay LOD represented by dashed lines. For body weight and oral swab vgRNA data, the placebo and bivalent low Al(OH)3 CpG 1018 groups contained 10 animals per group, while bivalent high Al(OH)3 group contained 8 animals. For viral load data, 8 animals per group were tested. Data was assessed by one-way ANOVA with Dunn's multiple comparisons to determine statistical significance. Body weight data is shown as SEM, vgRNA and Viral Load data is displayed as median±95% CI. vgRNA results for Placebo vs Bivalent with low Al(OH)3 were as follows: Day 1 (Beta P=0.0213, Delta P=0.0094), day 3 (Beta P=0.0002, Delta P=0.0001) and day 5 (Beta P=<0.0001, Delta P=<0.0001).

FIG. 7B. Bivalent SARS-CoV-2 vaccine candidate protected hamsters when challenged with Beta and Delta—Post-challenge body weight are shown. Assay LOD represented by dashed lines. For body weight and oral swab vgRNA data, the placebo and bivalent low Al(OH)3 CpG 1018 groups contained 10 animals per group, while bivalent high Al(OH)3 group contained 8 animals. For viral load data, 8 animals per group were tested. Data was assessed by one-way ANOVA with Dunn's multiple comparisons to determine statistical significance. Body weight data is shown as SEM, vgRNA and Viral Load data is displayed as median±95% CI. vgRNA results for Placebo vs Bivalent with low Al(OH)3 were as follows: Day 1 (Beta P=0.0213, Delta P=0.0094), day 3 (Beta P=0.0002, Delta P=0.0001) and day 5 (Beta P=<0.0001, Delta P=<0.0001).

FIG. 7C. Bivalent SARS-CoV-2 vaccine candidate protected hamsters when challenged with Beta and Delta—viral genomic RNA from oral swabs for Beta variant are shown. Assay LOD represented by dashed lines. For body weight and oral swab vgRNA data, the placebo and bivalent low Al(OH)3 CpG 1018 groups contained 10 animals per group, while bivalent high Al(OH)3 group contained 8 animals. For viral load data, 8 animals per group were tested. Data was assessed by one-way ANOVA with Dunn's multiple comparisons to determine statistical significance. Body weight data is shown as SEM, vgRNA and Viral Load data is displayed as median±95% CI. vgRNA results for Placebo vs Bivalent with low Al(OH)3 were as follows: Day 1 (Beta P=0.0213, Delta P=0.0094), day 3 (Beta P=0.0002, Delta P=0.0001) and day 5 (Beta P=<0.0001, Delta P=<0.0001).

FIG. 7D. Bivalent SARS-CoV-2 vaccine candidate protected hamsters when challenged with Beta and Delta—viral genomic RNA from oral swabs for Delta variant are shown. Assay LOD represented by dashed lines. For body weight and oral swab vgRNA data, the placebo and bivalent low Al(OH)3 CpG 1018 groups contained 10 animals per group, while bivalent high Al(OH)3 group contained 8 animals. For viral load data, 8 animals per group were tested. Data was assessed by one-way ANOVA with Dunn's multiple comparisons to determine statistical significance. Body weight data is shown as SEM, vgRNA and Viral Load data is displayed as median±95% CI. vgRNA results for Placebo vs Bivalent with low Al(OH)3 were as follows: Day 1 (Beta P=0.0213, Delta P=0.0094), day 3 (Beta P=0.0002, Delta P=0.0001) and day 5 (Beta P=<0.0001, Delta P=<0.0001).

FIG. 7E. Bivalent SARS-CoV-2 vaccine candidate protected hamsters when challenged with Beta and Delta—day 35 lung viral load are shown. Assay LOD represented by dashed lines. For body weight and oral swab vgRNA data, the placebo and bivalent low Al(OH)3 CpG 1018 groups contained 10 animals per group, while bivalent high Al(OH)3 group contained 8 animals. For viral load data, 8 animals per group were tested. Data was assessed by one-way ANOVA with Dunn's multiple comparisons to determine statistical significance. Body weight data is shown as SEM, vgRNA and Viral Load data is displayed as median±95% CI. vgRNA results for Placebo vs Bivalent with low Al(OH)3 were as follows: Day 1 (Beta P=0.0213, Delta P=0.0094), day 3 (Beta P=0.0002, Delta P=0.0001) and day 5 (Beta P=<0.0001, Delta P=<0.0001).

FIG. 7F. Bivalent SARS-CoV-2 vaccine candidate protected hamsters when challenged with Beta and Delta—day 35 nasal turbinate viral load are shown. Assay LOD represented by dashed lines. For body weight and oral swab vgRNA data, the placebo and bivalent low Al(OH)3 CpG 1018 groups contained 10 animals per group, while bivalent high Al(OH)3 group contained 8 animals. For viral load data, 8 animals per group were tested. Data was assessed by one-way ANOVA with Dunn's multiple comparisons to determine statistical significance. Body weight data is shown as SEM, vgRNA and Viral Load data is displayed as median±95% CI. vgRNA results for Placebo vs Bivalent with low Al(OH)3 were as follows: Day 1 (Beta P=0.0213, Delta P=0.0094), day 3 (Beta P=0.0002, Delta P=0.0001) and day 5 (Beta P=<0.0001, Delta P=<0.0001).

FIG. 8A. Hamster Serum RBD IgG and nAb Hamster IgG levels to the SARS-CoV-2 RBD were assessed at days 21 and 35 by ELISA. Endpoint titers were expressed as the reciprocals of the final detectable dilution with an OD above the cut-off value, which was defined as the average OD of the placebo. Placebo and Bivalent with CpG groups contained 10 animals each, while bivalent with Al(OH)3 contained 8 animals.

FIG. 8B. PRNT50 is defined as the highest dilution of serum that results in 50% reduction of plaque-forming units, sera from 5 animals from the placebo group were tested, and sera from 8 animals from each of the vaccine groups were tested. For all data in FIG. 8A, One-Way ANOVA with Dunn's multiple comparison was carried out to determine significance.

FIG. 8C. PRNT50 is defined as the highest dilution of serum that results in 50% reduction of plaque-forming units, sera from 5 animals from the placebo group were tested, and sera from 8 animals from each of the vaccine groups were tested. For all data in FIG. 8B, One-Way ANOVA with Dunn's multiple comparison was carried out to determine significance.

FIG. 9. Immunohistochemistry of SARS-CoV-2 Nucleocapsid in lungs of vaccinated hamsters challenged with Beta or Delta SARS-CoV-2 virus. Immunohistochemistry on lung tissue harvested 5 days post-challenge. Anti-SARS-CoV-2 nucleocapsid antibody is displayed in brown.

Table 1. Histopathology scores of infected vaccinated lung tissues. Scoring legend: Mild=1, Moderate=2 and Severe=3.

### DESCRIPTION OF THE INVENTION

A number of vaccines are currently available that use mRNA to generate a protein antigen using the human cell machinery. This mRNA sequence, once administered, is transcribed into a protein sequence within the host. Although effective, these RNA vaccines require careful handling, including a cold chain from manufacture, and the sole antigenic components with which the immune system is to respond.

It has been surprisingly discovered that mRNA vaccines, instead of being administered to a host, can be expressed in an established cell line and the antigenic portion collected and, preferably, conjugated with other materials. This eliminates the need for a cold chain and allows for the rapid development of highly effective vaccines against emerging variants.

By way of a non-limiting example, a conjugate COVID vaccine was prepared using the Covid trimer conjugated to CRM197 and associated with aluminum phosphate antigen mixed with CPG. The idea was to use a qualified cell line that could allow the same mRNA to get in the cells and generate the protein of interest. On purification the protein was conjugated to produce a vaccine. The importance is the quick change of the sequence as per the genetic mutation and following the same route using qualified cells. To make the protein by a routine method with a recombinant construct, the waiting period of creating a new cell line and qualifying the same could be several months of time which could be crucial for a pandemic. In contrast, the methods as described in this disclosure allow for the rapid development of the required modified protein as the mRNA system has already established. The conjugate vaccine has two advantages, which are not present in mRNA vaccines. The product does not need subzero temperatures for storage. Also, it provides long term immunity which mRNA does not provide. In addition, although mRNA is safe it still suffers from reactions which a conjugate vaccine does not. Using the innovativeness of the mRNA system to produce proteins using cell lines instead of human subject and creating a conjugate of such protein to make a more effective, better temperature storage and safer vaccine. Importantly, the methods disclosed herein allow for quicker changes to deal with genetic mutations to generate vaccines in time in Pandemics.

The following examples illustrate embodiments of the invention, but should not be viewed as limiting the scope of the invention.

### EXAMPLES

### Example 1 Method to Produce a Vaccine Using the Spike Protein of SAR-CoV2

The spike protein of SAR-CoV2 is the antigenic portion that is encoded within a number of mRNA vaccines. To maximize the potential benefit conferred by the S-2P antigen, methods of immunogenicity enhancement have been developed. Conjugation of bacterial polysaccharides to carrier proteins is a potent method to enhance immune responses to weakly immunogenic bacterial antigens, especially in young children. Conjugate vaccines prime for memory responses, resulting in long-lived immunity in adults and children alike. Although these techniques have traditionally been used to access T-cell mediated immunity in bacterial vaccines, including pneumococcal, meningococcal, typhoid, and haemophilus influenza B vaccines, protein-protein conjugation also benefits protein subunit viral vaccines, as shown for influenza and COVID. S-2P was conjugated to CRM197 via a platform conjugation technology to form highly immunogenic antigens. CRM197 is a non-toxic mutant of diphtheria toxin, widely used in commercial vaccines to prevent infectious diseases and enhance immunogenicity and an ideal carrier protein for conjugate vaccines because it is enzymatically inactive due to a single amino acid substitution (G52E) and possesses a well-established safety profile in clinical settings.

To further improve S-2P immunogenicity secondary adjuvants were implemented. The emergence of Toll-Like Receptor (TLR) agonists as adjuvants has allowed vaccine manufacturers to both enhance and target immune responses to the desired adaptive outcome. TLR agonists activate signaling pathways that lead to defined immune profiles, depending on the binding agonist. A TLR 7/8 stimulant was tested with aluminum hydroxide to determine if it resulted in elevated IgG and neutralizing antibodies compared to aluminum hydroxide alone. 3M-052 elicited promising results, however rapid approval is desired, so TLR agonist CpG 1018 was utilized in subsequent studies since it has similar downstream advantages to 3M-052 and had been included in previously approved vaccines.

Initial studies were performed with Wuhan S-2P in mice to determine if conjugation and dual adjuvants could lead to a highly immunogenic vaccine candidate, compared with S-2P protein with aluminum hydroxide alone. Following this study, B.1.351 (Beta) and B.1.617.2 (Delta) variants were peaking worldwide, causing increased transmission, disease severity, mortality, and immune evasion. It was evident that these strains would give rise to newer variants of unknown virulence, transmissibility, and immune evasion. Therefore, Beta and Delta variant S-2P proteins conjugated to CRM197±CpG 1018 were tested in mice and later hamster studies with challenge.

Production of broadly protective conjugate vaccines is expensive for LMIC using traditional methodologies, particularly when production occurs in high income nations such as the United States. To navigate this challenge, a highly automated GMP production plant that minimizes human labor was designed in-house and subsequently built to address global mortality attributed to pneumococcal disease. The vaccine described herein can be generated at large scale using this plant, allowing affordable production of a broadly cross-protective vaccine, minimizing the need for frequent boosters and vaccine modifications. Alternatively, an equivalent GMP production plant could be built in areas of highest need, allowing region-specific pathogens to be addressed locally, with even lower production costs. Such technology is not limited to COVID-19 and can be applied to disease both current and prospective.

### Example 2 Beta and Delta S-2P Expression in CHO2353 Cells, Purity, and Characterization

Due to the rapid acceleration of the pandemic, purified recombinant, soluble, trimerized S-2P spike proteins were obtained to test conjugation and formulation in animal studies. Stable CHO2353 pooled cells expressing Beta and Delta S-2P conjugates were also acquired, and development work was carried out in parallel to the animal studies, to adapt expression and purification techniques to meet project requirements.

The cell growth process was adapted from a method using CHO2353 stable pools, a fed-batch process, and a cumate induction system for S-2P protein expression that was developed. To optimize the production of both SARS-CoV-2 recombinant Beta and Delta S-2P proteins, the induction parameters were adjusted as it was determined for both that the overall level of expression stabilized after day 8 of induction for both Beta and Delta S-2P protein expression.

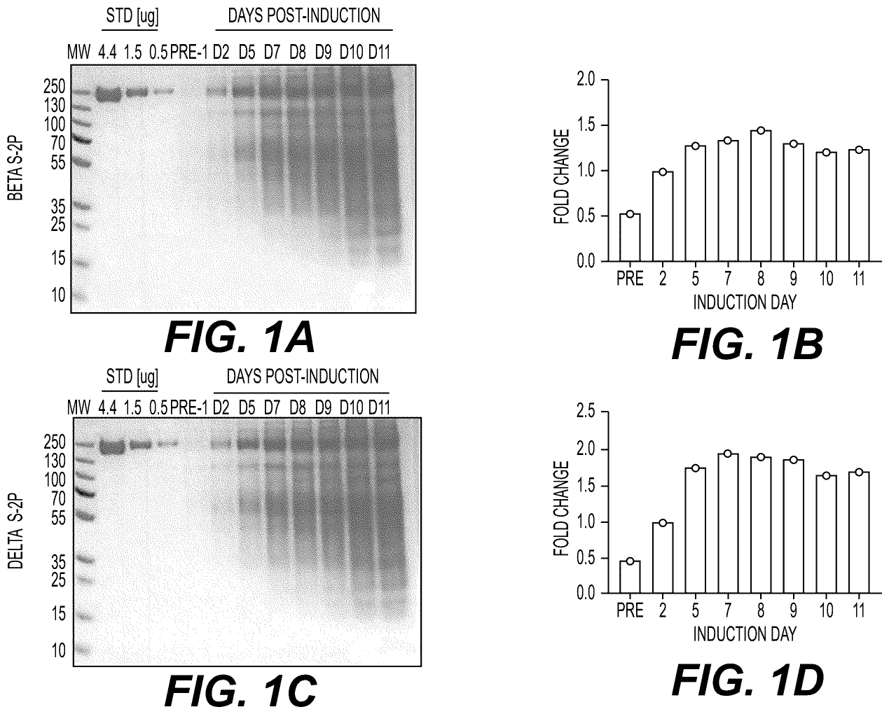

FIGS. 1A and 1C displaythe resulting SDS-PAGE gels and FIGS. 1B and 1D shows graphic representation of the change in expression levels versus time of induction. This adjustment 125led to a growth time of 17-20 days from time of inoculation to harvest.

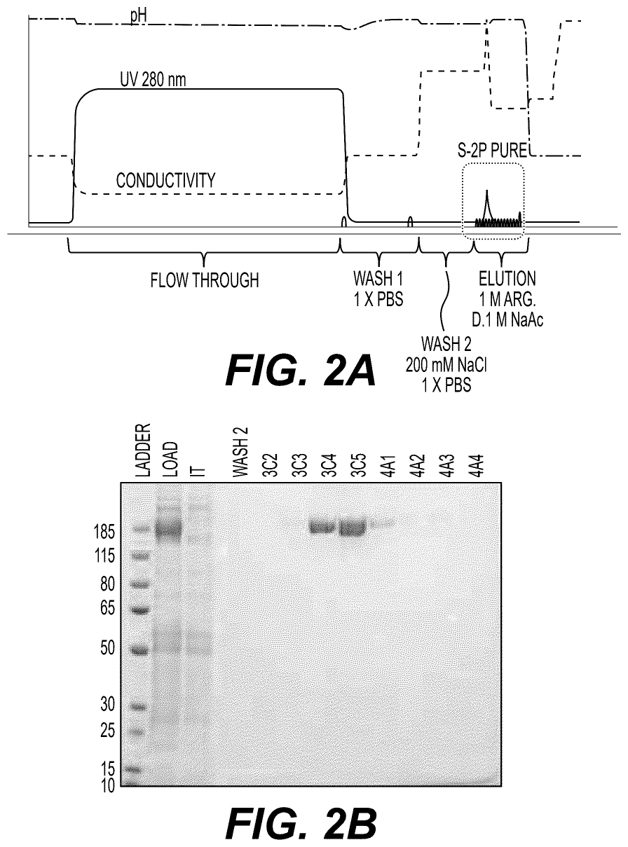

The downstream process was developed to purify SARS-CoV-2 S-2P proteins from other impurities. An experimental affinity chromatography resin was obtained: NGL Covid-19 Spike Protein AR 2.0. This resin was designed to bind the receptor binding domain (RBD) of the SARS-CoV-2 protein and subunits containing the RBD. Resin was equilibrated with 1X PBS, pH 8.0. Bound material was washed in two steps with 1X PBS, pH 8.0 (Wash 1), followed by 200 mM NaCl, 1X PBS, pH 8.0 wash (Wash 2) to remove impurities, and the S-2P protein was eluted using 0.1 M sodium acetate, 1 M arginine, pH 5. Fractions containing S-2P protein were pooled, further processed, and evaluated by SDS-PAGE gel. A representative chromatogram of the AKTA purification of Beta S-2P is shown in FIG. 2A, and correlates to the fractions represented in the SDS-PAGE gel in FIG. 2B. Elution fractions were pooled, buffer-exchanged, and brought to 5% Sucrose, 1X PBS, pH 7.4, for stabilization and long-term storage at −80° C., then sterile filtered.

The Beta and Delta S-2P manufacturing process was optimized to produce a stable, high quality drug substance for conjugating to CRM197 to form the vaccine antigens. The adjustment of these parameters minimized processing time, eliminated wasteful steps, and optimized yield while retaining a high level of purity. A reproducible and efficient method was developed resulting in good S-2P yields and >95% purity.

### Example 3 Conjugation and TLR-Agonist Improve Immunogenicity in Mice

To assess if conjugation of S-2P to CRM197 and addition of 3M-052 enhanced immune responses compared to adjuvanting with aluminum hydroxide alone, an accelerated proof of concept study was performed. Mice received two doses of each vaccine candidate, 14 days apart. Sera was collected and assessed for binding IgG and RBD-neutralizing antibodies. Vaccine candidates tested were: (1) 5 μg Wuhan S-2P and 12.5 μg aluminum hydroxide (Protein+Al(OH)3) (2) 5 μg Wuhan S-2P-CRM197 conjugate and 12.5 μg aluminum hydroxide (Conjugate+Al(OH)3) and (3) 5 μg Wuhan S-2P-CRM197 conjugate and 12.5 μg aluminum hydroxide and 0.2 μg 3M-052 (Conjugate+Al(OH)3+3M-052).

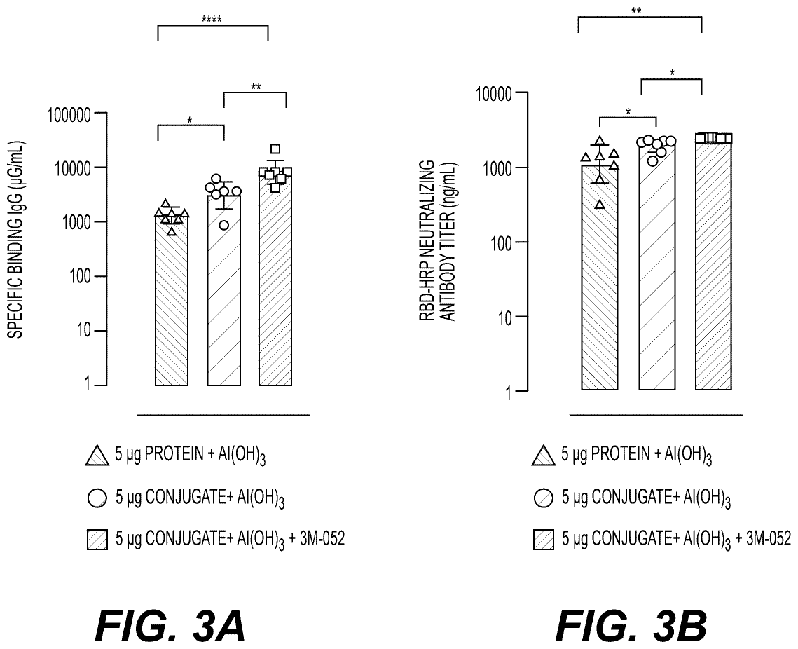

Two weeks post boost, animals vaccinated with Conjugate and Al(OH)3 had higher binding IgG compared to animals vaccinated with Protein and Al(OH)3.The addition of 3M-052 led to even higher titers than those achieved with only conjugation. The specific binding IgG levels between protein+Al(OH)3 and conjugate+Al(OH)3+3M-052 were significantly higher (P=0.0006) (see FIG. 3A). Similar results were observed for RBD-HRP Neutralizing Antibody 186 titers, with conjugation and addition of 3M-052 significantly improving RBD-HRP nAb titers (P=0.0012) (FIG. 3B). Consequently, subsequent studies focused on S-2P conjugates with and without secondary adjuvants.

### Example 4 Bivalent vs Monovalent Vaccine Immunogenicity in Mice

As new variants of the SARS-CoV-2 virus continued emerging, a vaccine targeting more relevant VOC than Wuhan was developed. Bivalent conjugate vaccines were compared to monovalent conjugate vaccines. Beta S-2P—CRM197 conjugates were combined with Delta S-2P CRM197 conjugates to form a bivalent vaccine, (Bivalent). Additionally, Beta conjugates and Delta conjugates were each tested independently, (Beta-only and Delta-only respectively). Each vaccine candidate (Bivalent, Beta-only and Delta-only) was tested with each of the following adjuvants: 1) 375 μg aluminum hydroxide (Low Al(OH)3); 2) 750 μg aluminum hydroxide (High Al(OH)3); 3) 375 μg aluminum hydroxide+10 μg CpG 1018 (Low CpG) and 4) 375 μg aluminum hydroxide+25 μg CpG 1018 (High CpG). The TLR-9 agonist CpG 1018 was used as a secondary adjuvant to support a streamlined path to approval.

Mice received two intramuscular doses of each vaccine candidate 21 days apart. PBS served as a placebo. Sera was collected at days 35 and 49 and assessed for Binding IgG to Beta S-2P and Delta S-2P. nAb titers to Beta, Delta, Omicron BA.1, Omicron BA.2, Omicron BA.4/5, and Omicron XBB 1.5 were measured using pseudovirus neutralization assays. Due to limited sera and reagents, Omicron BA.4/5 variants were measured in day 35 sera, and Omicron XBB 1.5 was measured in day 49 sera. At the time of testing Omicron was the current VOC. Omicron BA.4 and Omicron BA.5 share the same spike protein and could be tested using a mutual pseudoparticle. Delta-only High Al(OH)3 samples were not tested for nAb to Omicron BA 4.5 due to insufficient reagents.

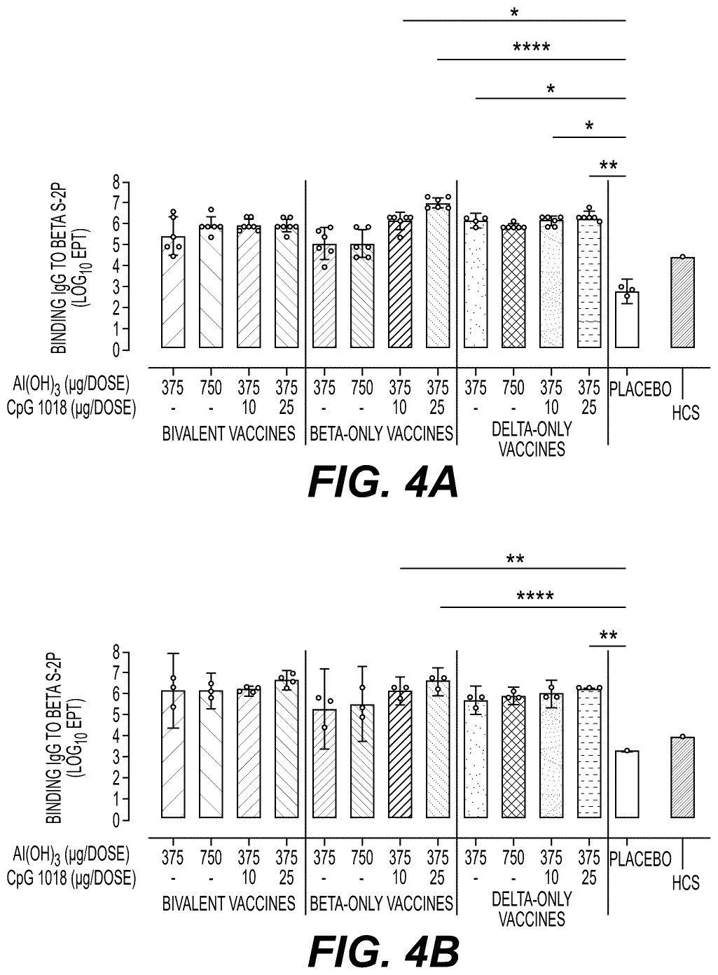

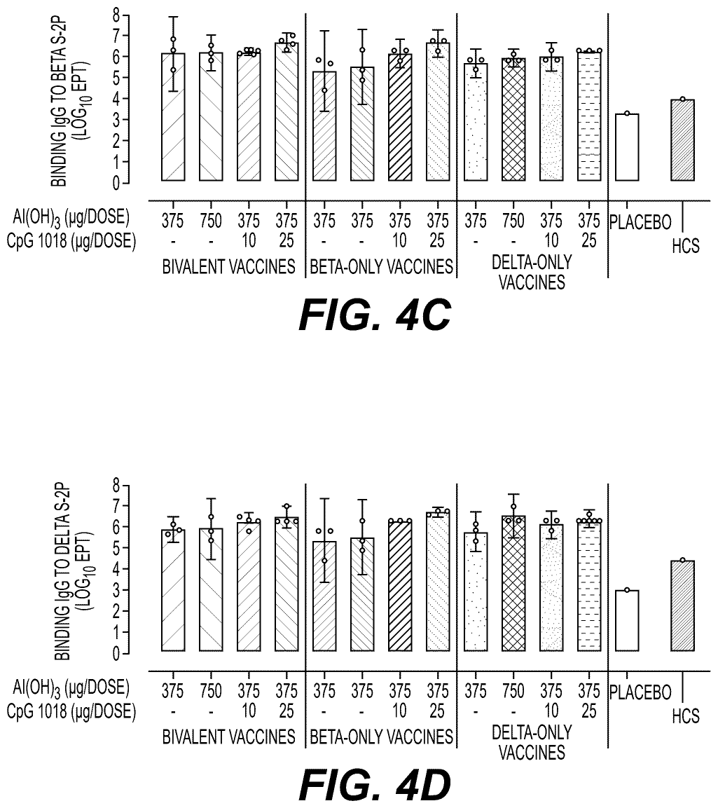

A pool of high titer human anti-SARS-CoV-2 immunoglobulin Human 222 Convalescent Serum 20/150 (HCS) was included in the assays as a comparator. Regardless of immunogen, the Beta-only high CpG generated the highest binding IgG titers to both Beta and Delta S-2P at day 35 (P=<0.0001) and day 49 (P=<0.0001) compared to placebo (FIGS. 4A-4D). The titers of the Bivalent High CpG were approaching those of the Beta-only High CpG groups by day 49; however, this titer was reached two weeks earlier for the Beta-only High CpG candidate.

All vaccine candidates elicited nAb capable of cross protection to all variants tested, to varying degrees. There was no significant difference in the titers obtained for each variant tested for the Bivalent Low Al(OH)3, Bivalent High Al(OH)3, Beta-only Low CpG, Beta-only High CpG, and all Delta-only vaccines. The titers from Delta-only vaccines were lower across the board, so were excluded from further investigation. Surprisingly, the highest titers were from the Beta-only High CpG vaccine rather the Bivalent candidates, which were expected to be superior based on having two antigens and a total dose of 10 μg compared to the monovalent vaccines that were 5 μg per dose. Statistical analysis could not be applied to the day 49 sera, as there were only 3-4 samples per group. This was due to half of the mice in each group undergoing splenectomies at day 35, and therefore could not provide sera at day 49. Nevertheless, the data shows robust titers to all variants including Omicron XBB 1.5. Although these titers may not be equivalent to those seen for other variants, and are from a small number of animals, this still demonstrate a degree of protection against variants that emerged long after the antigenic strains

### Example 5 Variant Timeline

To apply theoretical longevity to the protection against variants inferred by the mouse nAb data, a timeline of worldwide cases of each variant that was tested for murine nAb was generated using data from https://ourworldindata.org/grapher/covid-variants-area using data from GISAID via CoVariants.org. Beta variant emerged in August 2020 and Delta in October 2020.

The nAbs produced from these vaccine candidates neutralized variants that were still in circulation three years after the antigenic strain emerged, at minimum.

Serological data from mice studies indicated that all vaccines offered protection from disease, with the Bivalent and the Beta-only with High CpG candidates the most promising. To confirm, efficacy study in hamsters was performed with the Bivalent vaccine candidate±CpG 1018. The Beta-only High CpG candidate was not included in the challenge because the rapid evolution of the pandemic resulted in the challenge study being initiated prior to acquiring all the serological data in the previous mouse study. Therefore, the breadth of protection offered by the Beta-only High CpG candidate was not known at the time. Despite that, the following data prove the Bivalent candidate induced a potent immune response that protected hamsters from disease after challenge with live SARS-CoV-2 Beta and Delta virus.

Male Golden Syrian Hamsters received two intramuscular doses of each vaccine candidate or placebo 21 days apart. Vaccine candidates were Bivalent High Al(OH)3 (5 μg Beta Conjugate+5 μg Delta Conjugate+750 μg aluminum hydroxide) and Bivalent Low Al(OH)3 and CpG (5 μg Beta Conjugate+5 μg Delta Conjugate+350 μg aluminum hydroxide+100 μg CpG 1018).

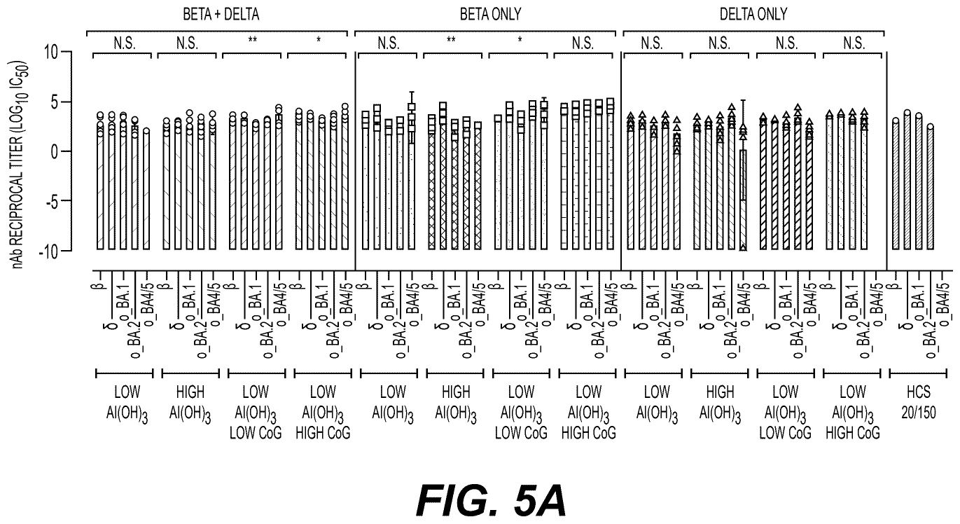

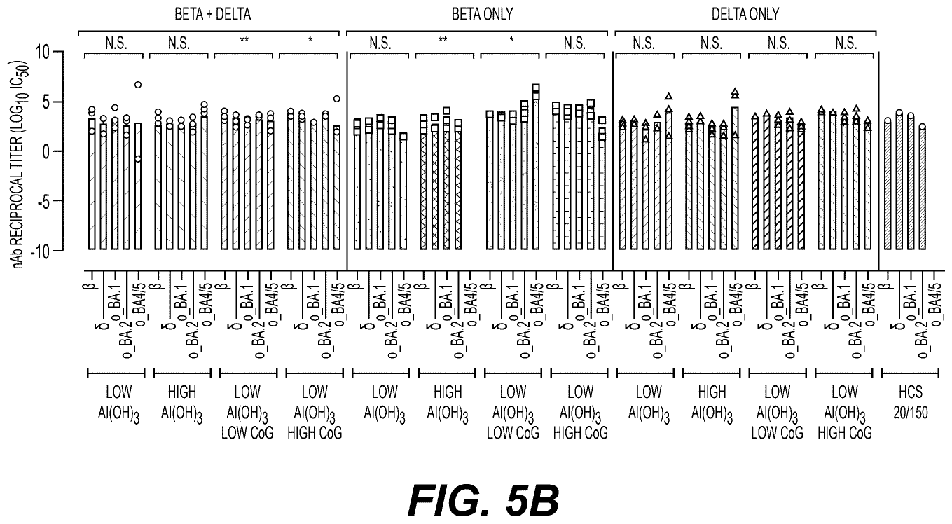

Sera was collected at days 0, 21, and 35 and assessed for RBD-binding IgG and Plaque Reduction Neutralization Test (PRNT50) (FIG. 5A). At day 35, two-weeks after the second dose, animals were intranasally challenged with 1×105 PFU/animal of either SARS-CoV-2 Beta variant or Delta variant. Body weight was measured for 5 days post-challenge. Oral swabs were collected 1-, 3-, and 5-days post challenge to determine viral load. Animals were euthanized 5-days post challenge, and necroscopy was carried out. Lungs and nasal turbinate were harvested for plaque assay and viral genomic RNA (vgRNA) analysis. Lungs were harvested from a subset of animals for histology (n=2/group) and immunohistochemistry to the SARS-CoV-2 nucleocapsid.

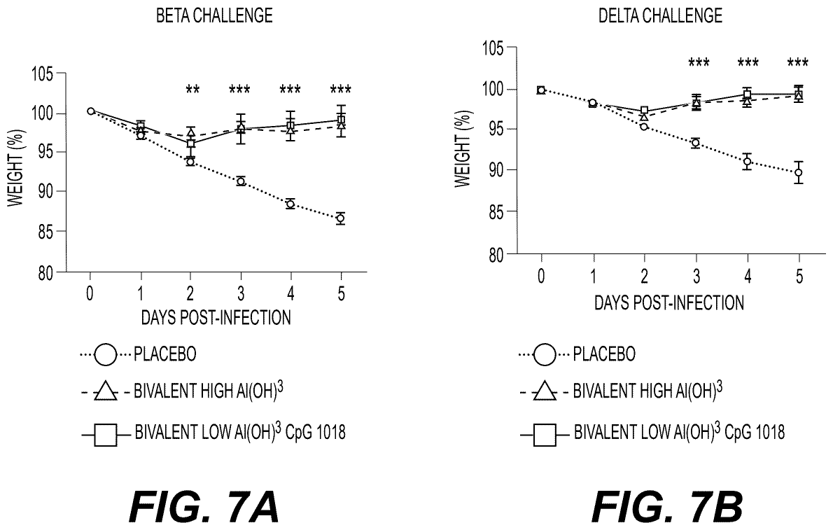

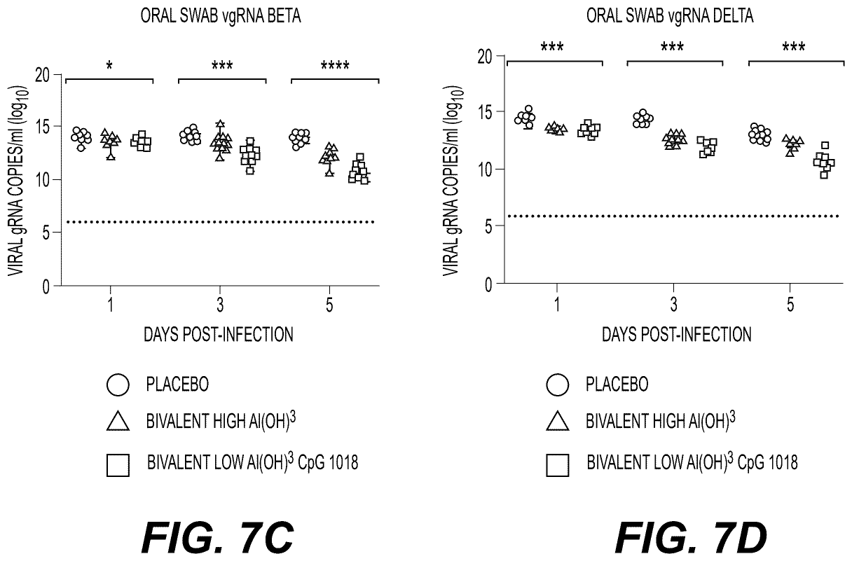

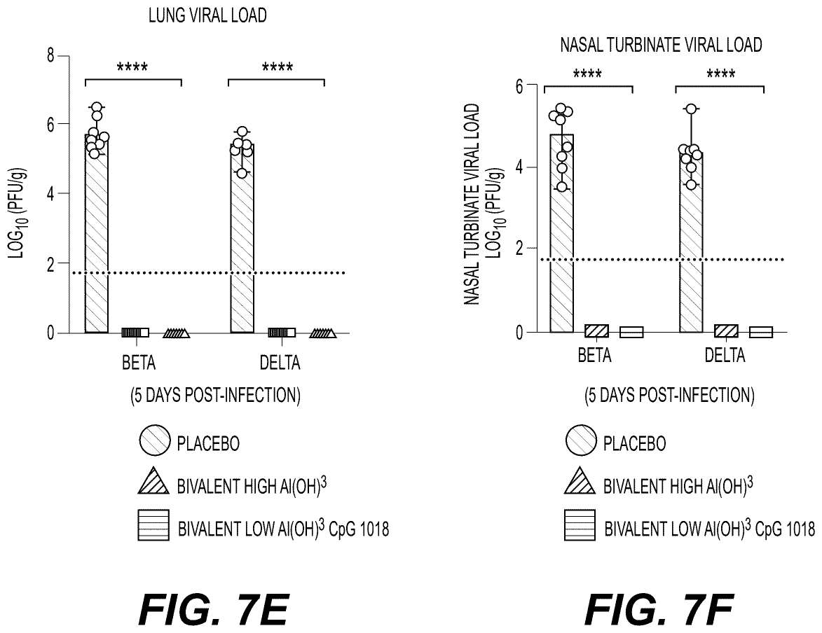

Body weights of vaccinated animals decreased to day 2, then returned to starting weight by day-5 post challenge (see FIGS. 7a and 7b). Animals in the placebo group continued losing weight to day 5 when the study was terminated. The virus variant used for challenge did not impact the pattern seen in body weight. Additionally, there was no difference observed between the body weight responses in animals vaccinated with Bivalent High Al(OH)3 compared to Bivalent Low Al(OH)3 and CpG.

Relative to placebo, vaccinated animals showed significantly lower vgRNA levels in oral swabs taken on days 1, 3, and 5 post challenge. Bivalent with Low Al(OH)3 and CpG group had the lowest vgRNA overall, which decreased the fastest signifying an efficacious vaccine.

At 5 days post-challenge, hamster lungs and nasal turbinate were collected and tested for viral load by plaque assay. For both vaccine groups there was no virus detected in the lung or nasal turbinate, regardless of variant used for the viral challenge. Conversely, the placebo group exhibited high levels of live virus in the sampled tissues.

Hamster binding IgG titers to the SARS-CoV-2 RBD were assessed and show that both vaccine candidates induced high levels of IgG after a single dose of vaccine. The IgG levels at Day 21 were higher in the CpG adjuvanted group, however by day 35 the IgG levels were equivalent between groups. Similar results were observed for PRNT50 tests to Beta and Delta variants, with high levels of nAb to both variants seen by day 35.

Hematoxylin and Eosin (H&E) staining was carried out on lung tissue collected at necroscopy from the Naïve, Placebo and Bivalent Low Al(OH)3 and CpG groups, and assessed. Pathology scores indicated in Table 1. H&E staining was not performed on the Bivalent High Al(OH)3 group. Challenge with live virus in the Placebo group led to a range of lung pathologies, and overall cytopathic effect. Challenge with Delta variant led to more severe pathologies than challenge with Beta. The vaccinated group showed only mild pathology, except for moderate Alveolar Histocytes for one animal challenged with Delta variant. Overall, no cytopathic effect was observed for vaccinated animals challenged with either Beta or Delta SARS-CoV-2, conversely animals in the Placebo group displayed clear cytopathic effect.

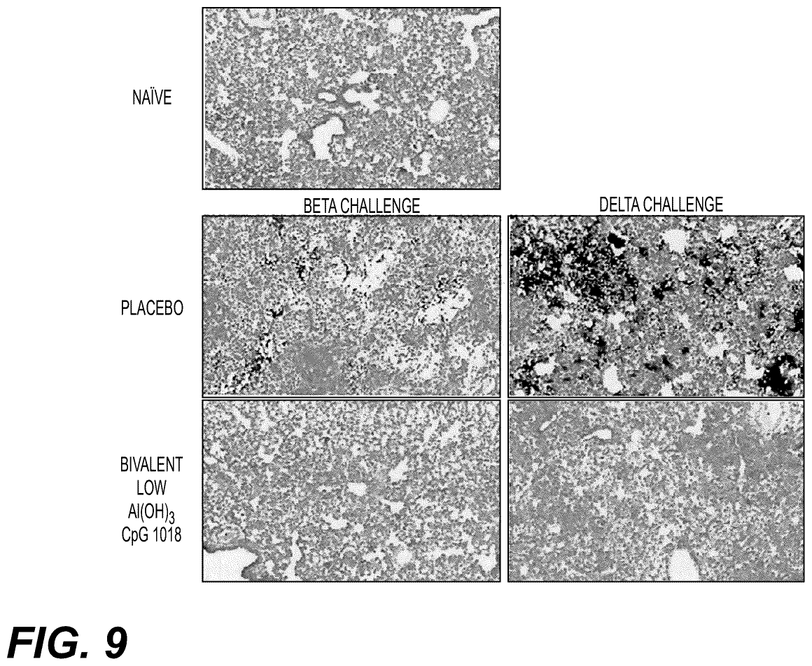

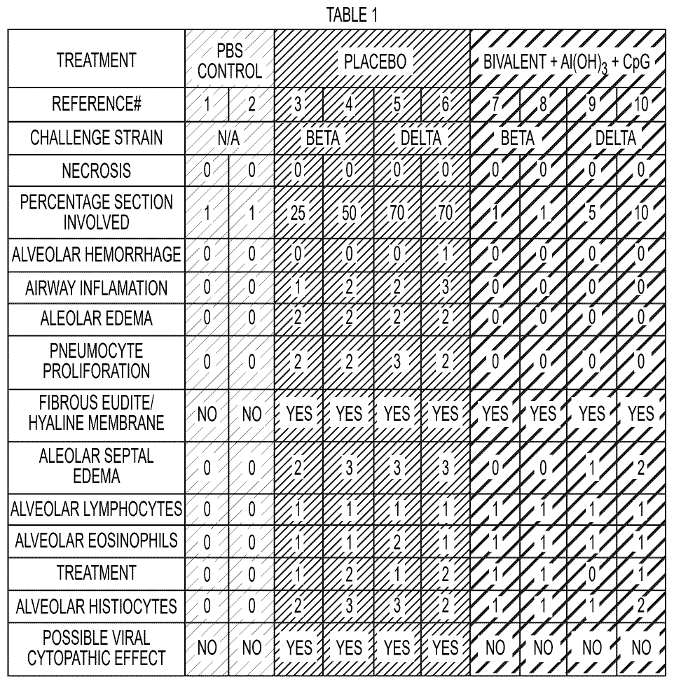

Immunohistochemistry was performed on lungs harvested 5 days post-challenge. Tissue sections were stained with anti-SARS-CoV-2 nucleocapsid antibody, shown in brown. Representative images of each group can be seen in FIG. 9. There was distinct staining to the SARS-CoV-2 virus in the lung tissue of the Placebo group, particularly the animals challenged with the Delta SARS-CoV-2 variant. In contrast, hamsters that received Bivalent Low Al(OH)3 and CpG vaccine had no staining to SARS-CoV-2 nucleocapsid, comparable to the unvaccinated and unchallenged naïve group. This data is consistent with the viral load data in FIG. 7, where no virus was detected in the lungs of vaccinated animals by plaque assay.

Together this data shows that S-2P conjugates vaccines with aluminum hydroxide with and without CpG 1018 induce robust immune responses and lead to protection from disease in animals.

Demonstrated here are highly immunogenic protein-protein SARS-CoV-2 conjugate vaccines that induce nAb capable of inhibiting VOC that emerged long after the antigenic strains, and effectively protect hamsters from disease. This vaccine is generated using our platform conjugation methods and is amenable to large-scale highly automated production with relatively low manufacturing costs, making it appealing for LMIC markets. Similar conjugation strategies are being utilized for several vaccines in our development pipeline, including a 25-valent pneumococcal vaccine, a Group B streptococcus vaccine, and a Shigella vaccine.

Limitations of mRNA vaccines to SARS-CoV-2 include cost,46 rapidly-waning immunity, and poor stability. Compared to mRNA vaccine manufacture, protein-based conjugate vaccine production is well-established, less expensive, and the resulting drug substances possess far greater stability profiles. The drug substance was shown to have a robust stability profile,16,18 with storage and transport anticipated to be at 2°—8° C., which is far more accessible than the prohibitive cold-chain requirements of mRNA vaccines.

One clear advantage of mRNA vaccines is that they can be produced quickly compared to protein subunit vaccines, with Moderna's mRNA SARS-CoV-2 vaccine being administered to first patient in first-in-human trials within 10 weeks of the genetic sequence being available. By utilizing the CHO2353 stable pool platform for antigen expression, GMP cell pooled banks can be produced and scaled up within 8-weeks. Following this, protein expression, downstream processes, and quality control can take an additional 8 weeks, meaning preclinical material can be available 16 weeks from the moment the pathogen sequence is available. Should regulatory guidelines be altered to allow pooled CHO pools for early-phase clinical material, then this timeline would be applicable to clinical drug products, minimizing the advantage that mRNA vaccines have compared to protein-conjugate vaccines.

The S-2P-CRM197 conjugate vaccines described herein led to potent immune responses in mice and hamsters and prevented infection in hamsters. The vaccine candidates were protective when adjuvanted with aluminum hydroxide alone; however, the addition of TLR 7/8 or 9 agonists resulted in superior immunogenicity. Additionally, these secondary adjuvants resulted in a narrower range of immune responses, indicating that all animals that received TLR agonist adjuvants generated equally high titers. The switch from 3M-052 in the proof-of-concept study to CpG 1018 in the subsequent studies was, at the time CpG 1018 was already approved in existing vaccines. Both 3M-052 and CpG 1018 were comparable in their ability to enhance the immunogenicity of the vaccines.

The Bivalent vaccine was considered the superior candidate, hence the accelerated path to challenge testing in hamsters. Surprisingly, the Beta-only High CpG 1018 candidate induced the highest serum IgG and nAb titers in mice and led to broad cross-protection against all variants tested, including Omicron BA.4 and Omicron BA.5 which emerged long after the Beta variant. This is in stark contrast to first generation mRNA vaccines, that did not show equal neutralizing antibody titers to variants when tested using similar methods. Although studies addressing long-term serological titers and memory immune responses were beyond the scope of this project, the memory responses exhibited in other bacterial and recently viral conjugate vaccines suggests that these vaccines could potentially induce similarly long-lasting protection from disease with the added benefit of broad cross protection to variants.

Second generation monovalent mRNA vaccines based on the Beta spike protein did not elicit equivalent nAb to all VOC when tested with pseudovirus indicating that they are better suited as a booster vaccine, but not appropriate as a primary vaccine series. Although different panels of pseudoviruses were used, Beta-only mRNA vaccines administered as a primary series, were unable to elicit the degree of cross protection to VOC displayed here, highlighting the quality of the neutralizing antibodies produced in response to the Beta-only High CpG conjugate vaccine.

This data shows that the Beta-Delta conjugate vaccine with CpG 1018 is suitable as a standalone vaccine, and that the conjugation methods and addition of secondary adjuvants employed here yield a highly immunogenic vaccine that induce a response capable of withstanding challenge with live virus.

The merits of conjugating bacterial polysaccharides to carrier proteins are well documented. It was shown herein that conjugating S-2P to CRM197 leads to improved immunological markers than unconjugated equivalents, which is enhanced further with dual adjuvants. The highly effective neutralizing antibodies are strong indicators of protection, and the broadly cross protective nAb in preclinical data suggests that the Beta-only conjugate with High CpG 1018 had outstanding potential as a vaccine. A similar style vaccine comprised of the SARS-CoV-2 RBD conjugated to tetanus toxoid was developed and used in large-scale vaccination campaigns in Cuba, and provided long-lasting immunity, when administered as a heterologous 3-dose regime, indicating that the immunological longevity of conjugate vaccines is not limited to polysaccharide protein conjugates.

The Beta-only High CpG vaccine candidate serves as a robust primary vaccine that offered protection against a wide range of variants, including Omicron, which evaded protection from numerous other vaccines. Furthermore, the broad cross-protection against VOC, despite mutations that occurred years after the antigen emerged, coupled with the immunological longevity imparted by conjugate vaccines highlights the unique properties of this vaccine. Common cold like viruses such as SARS-CoV-2, Influenza and Respiratory Syncytial Virus replicate in the respiratory mucosa, have relatively short incubation periods, do not cause marked viremia, and do not elicit long-term protective immunity from natural infection. Similarly, vaccines targeting these diseases do not provide durable protection or control of disease. Frequent boosters with COVID-19 mRNA vaccines have been necessary to maintain immunity over time and against evolving VOC, while lessened protection to variants that emerged after the mRNA vaccine antigens led to frequent updates to vaccine design. These factors are financially burdensome and not ideal for globally accessible vaccines. The method described herein can be applied to the ongoing pursuit of universal vaccine to SARS-CoV-2 and other respiratory viruses that require additional modulations to boost their immunogenicity.

mRNA vaccine platforms allow accelerated development and deployment of vaccines in emergencies, and as a result are attributed with saving millions of lives during the COVID-19 pandemic. When assessing them for cost, stability, and long-term protection against evolving viruses, mRNA vaccines have some limitations. By leveraging our proprietary conjugation techniques together with TLR agonist adjuvants and applying them to a protein subunit antigen that can be rapidly produced at high levels, a vaccine was produced with enhanced immunogenicity capable of incredibly broad cross-protection against VOC including those that were in circulation up to three years after the antigenic strains first appeared. These techniques can be pursued as a potent mechanism to enhance immunogenicity and length and breadth of protection offered by protein-based vaccines for viruses both current and yet to emerge.

### Example 6 Methodology

Growth and Expression of Beta and Delta S-2P in CHO2353 cells

Stable CHO2353 pools expressing Beta and Delta S-2P were licensed from National Research Council Canada (Montreal, Quebec, Canada). The BalanCD CHO media platform Growth A, Feed 4, Anti-clumping agent, Accumax, dextrose monohydrate, 10X PBS, glycerol, Halt Protease Inhibitor Cocktail, and Countess Automated Cell Counter and slides were purchased from Thermo Fisher Scientific (Waltham, MA, USA), a distributor of FUJIFILM Irvine Scientific (Santa Ana, CA USA). 4-isopropylbenzoic acid (Cumate) was purchased from Ark Pharma Scientific Limited (Wuhan, Hubei, China). Poloxamer 188 (KP188), L-methionine sulfoximine (MSX), and Benzonase were purchased from Millipore Sigma (St Louis, MO, USA). WaterSep Researcher24 Green 0.2 μm m-PES hollow fiber membrane was purchased from Sartorius (Göttingen, Lower Saxony Land, Germany).

CHO2353 pooled cells were grown using the BalanCD CHO Growth A media supplemented with 50 μM MSX, 0.2% KP188, and 0.1% Anti-clumping solution. Initial seeding was done at 3×105 cells/mL in a flask and was incubated at 37° C., 5% CO2, on an orbital shaker at 120 rpm, via subculturing to a viable cell density (VCD) of 5−10×106 cells/mL. Cell counting was performed using Accumax and an automated Countess system. After VCD was reached S-2P protein expression was induced and the culture was brought to 2 μg/mL Cumate, 125 μM MSX, and 5% 0.8× Feed 4. The incubation temperature for induction was decreased to 32° C., 5% CO2, on an orbital shaker at 120 rpm. A fed-batch production method was used in which Feed 4 and glucose were added at a rate of 2.5% v/v per day. Additional glucose was added to bring concentration up to 35 mM glucose. Induction continued until viability 70% was reached or 8±1 days. After expression, harvested cells were clarified via 0.2 μm tangential flow filtration (TFF). The clarified permeate was then treated with nuclease and protease inhibitor. The clarified, treated bulk was brought to 20% glycerol, aliquoted, and frozen at −80° C. until downstream processing.

### Protein Purification

A pre-commercial affinity chromatography resin was obtained: NGL Covid-19 Spike Protein AR 2.0 (Waltham, MA, USA). Resin was used in conjunction with an AKTA Avant 150 system from Cytiva Life Sciences (Marlborough, MA, USA). 1 M Dithiothreitol (DTT), 10X PBS, arginine, sodium chloride, sodium acetate, sucrose, 20 X NuPAGE MOPS SDS Running Buffer, 20X NuPAGE MES SDS Running Buffer, 4X NuPAGE LDS Sample Buffer, NuPAGE 4-12% Bis-Tris Protein Gels (1.0mm; 10 and 12 well), Invitrogen Bolt 4-12% Bis-Tris Mini Protein Gels (1.0 mm, 10-well), PageRuler Plus Prestained Protein Ladder, Pierce Modified Lowry Protein Assay, and SimplyBlue Safe Stain were purchased from Thermo Fisher Scientific (Waltham, MA, USA). 50 kDa and 100 kDa Amicon Ultra-15 spin filters, and 0.2 μm Steriflip were purchased from Millipore Sigma (St Louis, MO, USA). WaterSep Discover12 Green 300kDa m-PES hollow fiber membrane was purchased from Sartorius (Göttingen, Germany).

The affinity resin was equilibrated with 1X PBS, pH 7.4. Bulk material was removed from storage at minus 80° C., thawed at 4° C., brought to pH 8.0, and loaded onto the resin using the AKTA Avant 150 system. Bound material was washed with 1X PBS, pH 8.0, followed by 1 M NaCl, 1X PBS, pH 8.0. The protein was then eluted using 0.1 M sodium acetate, 1 M arginine, pH 5. The eluted protein was concentrated with a 50 kDa or 100 kDa Amicon Ultra-15 spin filter and discontinuously diafiltered 10-fold into 1X PBS, pH 7.4. For larger batch sizes, a 300 kDa hollow fiber TFF membrane was utilized for concentration and 10-fold continuous diafiltration with 1X PBS, pH 7.4. The concentrated S-2P protein was brought to 5% sucrose, sterile filtered using a 0.2 μm Steriflip, aliquoted, and frozen at −80° C.

Concentration was determined with the Pierce Modified Lowry Protein 573 Assay using BSA standards. Protein purity was evaluated by denaturing the protein with 0.1 M DTT and utilizing the NuPAGE 4-12% Bis-Tris or Bolt 4-12% Bis-Tris Mini Protein Gel SDS-PAGE gel systems in 1X MOPS or MES buffer. The gel was run at 200 volts for 42 minutes, stained with SimplyBlue Safe Stain for 60 minutes, and destained in water prior to imaging.

### Conjugation

1-Ethyl-3-(3-dimethylaminopropyl)carbodiimide hydrochloride (EDC), N-hydroxysuccinimide (s-NHS), sodium periodate, sodium cyanoborohydride, and adipic acid dihydrazide (ADH) were purchased from Millipore Sigma (St Louis, MO). Amine-PEG-Carboxyl (NH2-PEG-COOH), and carboxylic acid PEG NHS (NHS-PEG-COOH) were sourced from Biochempeg (Changsha, Hunan, P. R China). MES, Tris-Hcl, NaCl, PBS, and 100 kDa MWCO dialysis membranes were purchased from Thermo Fisher Scientific (Waltham, MA, USA).

S-2P proteins (7 mg/mL) were prepared in PBS and adjusted to pH 5.1±0.2. EDC and s-NHS was dissolved in 0.1 M MES pH 5.00 and pH 6.10 respectively then added to proteins and activated for 10 minutes at room temperature pH 5.2±0.2. CRM-PEG-COOH linker was added to the activated proteins and incubated for 1 hour at pH 6.6±0.2. Conjugate was purified and buffer exchanged into PBS pH 7.2 using 100 kDa MWCO protein concentrators.

Wuhan-Hu-1 S-2P was sourced from Excellgene (Monthey Switzerland). Aluminum hydroxide was purchased from Croda (Frederikssund, Denmark). 3M-052 TLR agonist was a kind gift from 3M Drug Delivery Systems (St. Paul, MN). Dipalmitoylphosphatidylcholine (DPPC), cholesterol, and chloroform were purchased from Millipore Sigma (St. Louis, MO, USA). WFI, sodium chloride and 1X PBS were purchased from Thermo Fisher Scientific (Waltham, MA, USA). L-histidine and polysorbate-80 were from Avantor Sciences (Radnor, Pennsylvania, USA).

The liposomal adjuvant was prepared using modifications to the thin-film hydration method described in literature58. Briefly, the lipid stocks of Dipalmitoylphosphatidylcholine (DPPC), cholesterol, and 3M-adjuvant were made in chloroform. The ratio of 3M-052 adjuvant to lipids was 1:10 (w/w). The lipids were purged with N2 in a sterile test tube and the chloroform was allowed to evaporate. The thin dried lipid film that was formed was hydrated in WFI (Water for Injection) at 50° C. for 25 min. The liposomes were vortexed and sonicated at 50° C. for 15 min to form a homogenous suspension and stored at 2-8° C. until further use.

Final formulations consisted of 5 μg S-2P protein (Wuhan) alone and 12.5 μg aluminum hydroxide, 5 μg S-2P conjugate (Wuhan) and 12.5 μg aluminum hydroxide, or 5 μg S-2P conjugate (Wuhan) and 12.5 μg aluminum hydroxide and 0.2 μg 3M-052.

### Beta and Delta S-2P Conjugate Mouse Study Formulation

Beta S-2P and Delta S-2P spike proteins were purchased from NRC. CpG 1018 was kindly donated by Dynavax (Emeryville, CA USA). Conjugates were formulated at 5 μg per antigen with either 10 μg or 25 μg of CpG-1018 and 375 μg aluminum hydroxide or 750 μg aluminum hydroxide alone.

Beta S-2P and Delta S-2P spike conjugate formulations consisted of 5 μg of each Beta and Delta antigen conjugate plus either 100 μg of CpG-1018 and 350 μg aluminum hydroxide, or 750 μg aluminum hydroxide alone. Formulations were carried out as per instructions provided by Dynavax. Aluminum hydroxide concentrations were selected based on potential human doses.

29×9-week-old female BALB/c mice were purchased from Charles River Laboratories and acclimatized for 7 days following arrival. All animals purchased were used in the study. Intramuscular inoculations of vaccine or placebo were administered to hind legs at days 0 and 14. Three vaccine candidates were tested 1) 5 μg Wuhan S-2P alone with 12.5 μg aluminum hydroxide, (n=7) 2) 5 μg Wuhan S-2P conjugated to CRM197 with 12.5 μg aluminum hydroxide (n=7), and 3) 5 μg Wuhan S-2P conjugated to CRM197 with 12.5 μg aluminum hydroxide and 0.2 μg 3M-052 (n=8). PBS served as a placebo (PBS, n=7). Animals were monitored daily and remained healthy throughout study. Blood was collected at days 0, 7, 14 and 28. For all bleeds, mice were anesthetized to surgical plane using a cocktail of Ketamine (2.5mg) and Xylazine (0.5mg), and blood was collected via retro-orbital vein (Days 0, 7 and 14) or jugular sever (Day 28). Clotted blood was centrifuged, and sera isolated. At the end, mice were anesthetized to surgical plane with a cocktail of Ketamine and Xylazine, exsanguinated, and then manual cervical dislocation ensured death.

7-week-old female BALB/c mice were purchased from Charles River Laboratories and allowed to acclimatize for 2-weeks following arrival. Animals were housed at 5 mice/cage with wood shavings and corn cob mixed bedding. Animals were arbitrarily assigned to the experimental groups; Animal facility employees were blinded to the contents of the vaccines. All purchased animals were used in the study. Intramuscular inoculations of vaccine or placebo were administered to hind legs at days 0 and 21. Sera was collected at Days 0, 21, 35 and 49. A subset of mice (3 per group) were exsanguinated at day 35 for splenectomies, reducing the group numbers by 3 for day 49 analysis (FIG. 5B).

12 vaccine candidates were tested, consisting of 3 antigens, each with 4 adjuvant combinations. The antigens tested were: Bivalent Beta and Delta conjugates (5 μg/conjugate/dose), Beta-only conjugate (5 μg/dose), and Delta-only conjugate (5 μg/dose). Each of these antigens were formulated with the following four adjuvant combinations: 1) 375 μg/dose aluminum hydroxide (Low Al(OH)3) (n=6 mice/group), 2) 750 μg/dose aluminum hydroxide (High Al(OH)3) (n=6 mice/group), 3) 375 μg/dose aluminum hydroxide+10 μg/dose (Low Al(OH)3, low CpG) (n=7 mice/group), and 4) 375 μg/dose aluminum hydroxide+25 μg/dose (Low Al(OH)3, high CpG) (n=7 mice/group). PBS served as a placebo (n=7). A total of 85 mice were used. For all bleeds, mice were anesthetized to surgical plane using a cocktail of Ketamine (2.5mg) and Xylazine (0.5mg), and blood collected via retro-orbital vein (Days 0, 21, 35). For final bleeds (Day 35 subset and Day 49), mice were anesthetized, and blood was collected via jugular sever. Clotted blood was centrifuged, and sera isolated. At the end of the study, mice were anesthetized to surgical plane with a cocktail of Ketamine and Xylazine, exsanguinated, then manual cervical dislocation 672 ensured death.

Immunoplates, PBS, HRP conjugated Goat-Anti-mouse IgG, HRP conjugated Goat-Anti human IgG, and 3,3 ,5,5-Tetramethylbenzidine (TMB) reagent were purchased from Thermo Fisher Scientific (Waltham, MA, USA). Bovine serum albumin (Fraction V) was purchased from Roche (Basel, Switzerland). Hydrochloric acid (HCl) was purchased from Millipore-Sigma (St Louis, MO, USA).

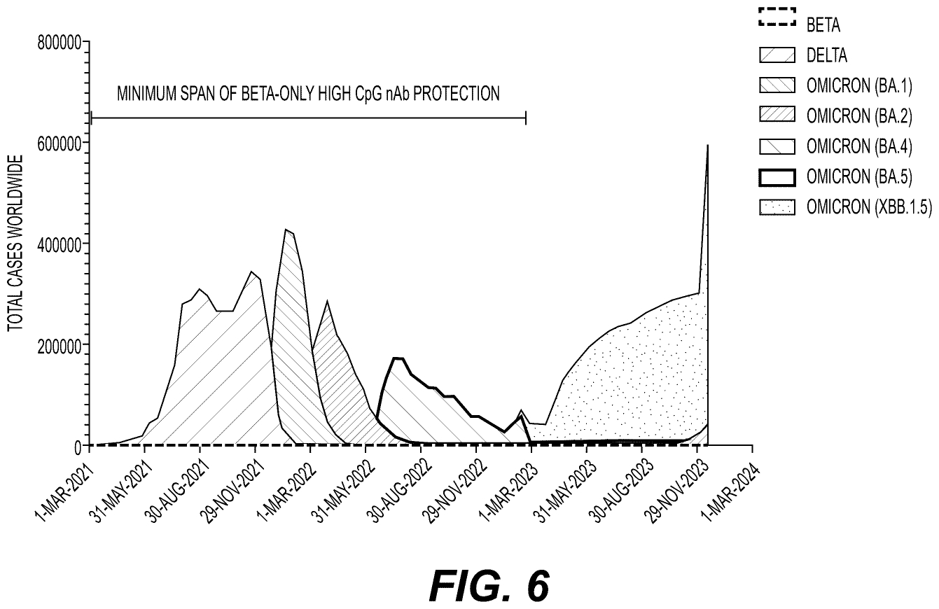

Sera was heat inactivated by incubating in a water bath set to 56° C. for 30 minutes. 96-well immunoplates were coated overnight with 1 μg/mL of antigenic protein diluted in PBS (Wuhan-Hu-1 S-2P for proof-of-concept mouse study, or Beta S-2P or Delta S-2P for bivalent vs monovalent mouse studies). Plates were blocked with 1% BSA in PBS, washed, and incubated with 11×3-fold dilutions of heat-inactivated antisera from individual mice in duplicate. Sera from all mice included in study were tested. Timepoint matched pooled sera from placebo group was included in each plate. Unbound sera were removed by washing and mouse IgG was detected with HRP conjugated Goat-Anti-mouse IgG. WHO Human Convalescent Sera of established binding units was used as a reference standard to quantitate the levels of binding IgG in proof-of-concept study only. Human IgG was detected with HRP conjugated Goat-Anti-human IgG. Unbound secondary antibody was removed with washing, and TMB reagent was added. The colorimetric reaction was stopped with 1 N HCl (FIG. 6).

Mouse Proof-of concept study: RBD-HRP Neutralizing Ab Test For mouse proof-of concept study, serum neutralizing antibodies (nAb) were measured using the SARS-CoV-2 Neutralization sVNT cPassTM Kit (GenScript, Piscataway, NJ, USA) as per manufacturer's instructions. Dilutions of mouse sera were incubated with Receptor Binding Domain (RBD)-HRP conjugate, then added to the ELISA plate coated with hACE2 receptor. Presence of nAb prevents RBD-HRP from binding to the hACE2 coated on the plate, and a color change does not occur. Sera was tested alongside dilutions of a neutralizing antibody standard purchased from the same manufacturer, allowing quantitative analysis of the neutralizing antibodies in the mouse sera. Sera from all mice per study group was tested.

Human Angiotensin-Converting Enzyme 2 (HEK-293T-ACE2) cells were obtained and cells maintained in Dulbecco's Modified Eagle's Medium containing 4 mM L-glutamine, 4500 mg per L glucose, 1 mM sodium pyruvate and 1500 mg per L sodium bicarbonate (DMEM High Glucose), supplemented with 10% fetal bovine serum (FBS) and antibiotics. All cell culture reagents were supplied by Gibco (Thermo Fisher Scientific, Waltham, MA, USA). Commercially available pseudovirus particles, encoding GFP and Luciferase reporters were purchased from Genecopoeia (Rockville, MD, USA) for all variants tested except Omicron BA 4/5, which was purchased from BPS Bioscience (San Diego, CA, USA). All pseudoparticles contained a lentivirus backbone and a SARS-CoV-2 Spike(S) protein which replaced the vesicular stomatitis G (VSV-G) envelope glycoprotein of the lentivirus.

The assay was adapted from published methods 59,60. Briefly, HEK-293T-ACE2 cells were seeded into columns 1-10 of white flat bottomed 96-well plates (Thermo Fisher Scientific, Waltham, MA, USA) at a density of 2×104 cells/well in 50 μL/well DMEM High Glucose supplemented with 10% FBS. Columns 11 and 12 receive media alone without 721 cells. Plates were incubated overnight at 37° C. with 5% CO2.

Heat inactivated sera from individual animals underwent 8×3-fold serial dilutions in antibiotic free DMEM High Glucose, starting at 1:20 dilution. SARS-CoV-2 Spike-Pseudotyped Lentivirus particles expressing spike proteins of VOC were diluted to 2×106 TU/mL in DMEM High Glucose. Equal quantities of sera dilutions and diluted pseudoparticles were mixed and incubated at 37° C. for 1 hour. For virus only controls, equal volumes of diluted pseudovirus and media were mixed and incubated. Incubated samples were added to the HEK-293T-ACE2 cells to give a final MOI of 5. Plates were incubated for 3 days at 37° C. with 5% CO2.

Bright-Glo Luciferase reagent (Promega, Madison, WI, USA) and assay plates were brought to room temperature. Equal volumes of luciferase reagent were added to the plates as per manufacturer's instructions. Luciferase activity was read on a plate reader. Half-maximal inhibitory concentration (IC50) analysis was carried out by calculating the maximum, which is the average relative light unites (RLU) of the Virus+Cells column and the minimum, which is the average RLU of the Media+Cells column. Virus only and Media only wells are expected to show background levels of RLU, as seen for Media only wells. In Graphpad Prism, data was normalized, using the calculated minimum as 0% and the calculated maximum as 100%.

Nonlinear fit [Inhibitor] vs. normalized response analysis was performed on the normalized data to determine the IC50.

Sera from all mice included in study were tested with the following exceptions, due to insufficient sera volumes. For day 35 nAb titer analysis, Omicron BA.2: Bivalent low Al(OH)3 n=5, Delta high Al(OH)3 n=5. Omicron BA.4/5: Bivalent low Al(OH)3 n=1, Bivalent high Al(OH)3 n=4, Bivalent low CpG n=6, Bivalent high CpG n=6, Beta-only low Al(OH)3 n=3, Beta-only high Al(OH)3 n=1, Beta-only low CpG n=4. For day 49 nAb titer analysis, Omicron XBB.1.5 Bivalent low Al(OH)3 n=2, Bivalent low CpG n=3, Bivalent high CpG n=3, Beta-only low Al(OH)3 n=1, Beta-only high Al(OH)3 n=0, Beta-only low CpG n=2, and Beta-only Hamster Studies

Golden Syrian hamsters, 7-8 weeks old (81-90 g) males, purchased from the Charles River Laboratories (Saint-Constant, Canada). Hamsters were vaccinated intramuscularly via the tibialis anterior with Bivalent High Al(OH)3 (5 μg Beta Conjugate+5 μg Delta Conjugate+750 μg aluminum hydroxide (n=8)) and Bivalent Low Al(OH)3 CpG (5 μg Beta Conjugate+5 μg Delta Conjugate+350 μg aluminum hydroxide+100 μg CpG 1018 (n=10) ) or Placebo (PBS (n=10)) at days 0 and 21 followed by a challenge with Delta or Beta variants at day 35, two weeks after second vaccination. Animals were arbitrarily assigned to each of the treatment groups. An additional 2 hamsters were assigned to the naïve group and received no treatment. A total of 30 animals were utilized in the study (FIGS. 7A-F).

Sera was collected at days 0, 21, and 35. Blood was collected via subclavian vein puncture into BD Microtainer Blood Collection Tubes, allowed to sit at 4° C. overnight, then centrifuged and serum collected. Animals were placed under anesthesia using isofluorane inhalation and maintained under anesthesia for the full duration blood was collected. End of study blood collection took placed immediately post euthanasia.

On day 35, hamsters were anesthetized by injection of Ketamine/Xylazine (90 kg/mg/8 kg/mg) and intranasally challenged with 1×105 plaque forming unit (PFU) of virus or 100 μl of sterile phosphate buffered saline (1X PBS) as control. Animals were weighed and monitored daily.

Animals were euthanized 5 days post-infection by isofluorane inhalation before exposure to CO2, then necroscopy performed. Lungs and nasal turbinate were harvested for plaque assay and viral genomic RNA (vgRNA) analysis. Lungs were harvested for histology and immunohistochemistry to the SARS-CoV-2 nucleocapsid. Histology was performed by a CRO with scoring carried out by a qualified pathologist.

The following SARS-CoV-2 isolates were used: SARS-CoV-2 Delta variant (hCoV-19/USA/MD-HP05647/2021; lineage B.1.617.2, BEI NR-55672), and Beta variant (hCoV-19/USA/MD-HP01542/2021; lineage B.1.351, BEI NR-55282) were obtained through BEI Resources, NIAID, NIH. Viruses were propagated on Vero E6 cells and quantified on Vero cells.

Sanger sequencing of the spike gene was carried out to confirm exact genetic identity to original isolate. Passage 3 or 4 virus stocks were used in all subsequent experiment.

Virus burden was quantified by plaque assay within the NRC CL-3 biocontainment facility. In brief, nasal turbinate and left lung were separately homogenized in 1 mL of 1X phosphate buffered saline (PBS). Clarified homogenate supernatant were serially diluted 1 in 10 in infection media (1X DMEM, high glucose media supplemented with 1X non-essential amino acid, 100 U/mL penicillin-streptomycin, 1 mM sodium pyruvate, and 0.1% bovine serum albumin). Virus was adsorbed on Vero cells for 1 hour at 37° C., inoculum was then removed and overlay media was added (1X infection media with 0.6% ultrapure, low-melting point agarose). The assay was incubated at 37°C/5% CO2 for 72 hours. After incubation, cells were fixed with 10% formaldehyde and stained with crystal violet. Plaques were enumerated and PFU was determined per gram of tissue. Procedure was carried out on 8 animals from each group.

Hamster Challenge Study: Plaque reduction neutralization tests Serum samples were inactivated at 56° C. for 30 min and stored on ice. The inactivated serum was serially diluted 1-in-2 and incubated with equal volume of 100 PFU of Beta or Delta virus at 37° C. for 1 hour. After incubation, adsorption of viruses was carried out for 1 hour at 37° C. on Vero cells, followed by removal of inoculum and cells overlaid with media as described above. The assay was incubated at 37° C./5% CO2 for 72 hours. Cells were fixed with 10% formaldehyde after incubation and stained with crystal violet. No serum, virus-only back-titer control was included along with naïve animal serum. PRNT50 is defined as the highest dilution of serum that results in 50% reduction of plaque-forming units. The 1-in-2 dilution of diluted serum to 100 PFU virus was included in the final calculation. The study was performed on 5 samples from placebo group, and 8 samples from each vaccine group.

Genomic viral RNA was quantified. Briefly, viral genomic RNA from oral swabs obtained from hamsters were extracted using Quick-viral RNA kit according to the manufactures (Zymo Research, Irvine, CA, USA). Luna Universal 821 One-step RT-qPCR kit was used to quantify viral genomic RNA (New England Biolabs, MA, USA) with primer/probe sets for the SARS-CoV-2 E gene (Forward: 5′ACAGGTACGTTAATAGTTAATAGCGT; Reverse: 5′ATATTGCAGCAGTACGCACACA, Probe: ACACTAGCCATCCTTACTGCGCTTCG 5′Fam 3′QSY-1). Standards were generated with known concentrations of viral RNA copies. 5 L of extracted RNA were run in duplicate on Applied Biosystems QuantStudio (Thermo Fisher Scientific, MA, USA) and results analyzed with Design and Analysis Software DA2 version 2.6.0.

RBD-specific IgG ELISA was conducted with vaccinated hamster sera following previously published protocols61. In brief, Nunc MaxiSorp flat-bottom 96 well plates (Thermo Fisher Scientific, Waltham, MA, USA) were coated with recombinant SARS-CoV-2 RBD-His recombinant protein (Sino Biological, Beijing, China) and incubated overnight at 4° C. Plates were then washed with 1X PBS containing 0.1% Tween-20 plates and blocked with 3% bovine serum albumin (IgG-Free). Hamster serum was serially diluted by 5-fold from 1:100 up to 1:1562500. Diluted sera were added to the plate and incubated for 1 hour at 37° C. Next, plates were washed with 1X PBS-T followed by addition of Peroxidase AffiniPure Goat Anti-Syrian Hamster IgG (H+L) (Jackson Immuno Research, West Grove, PA, USA) to each well and incubated at 37° C. for 1 hour. After the last wash with 1X PBS-T, 100 μL of Tetramethylbenzidine (TMB) substrate (Cell Signaling Technology, Danvers, MA, USA) was added to each well. After a two-minute incubation at room temperature, 100 μL of Stop solution (Cell Signaling Technology, Danvers, MA, USA) was added and absorbance was measured at 450 nm. Inhibitory dilution 50 (ID50) was calculated using non-linear regression analysis. Analysis was performed for all animals in each study group.

All four lobes of the right lung of infected hamsters were isolated and immersed in 10% neutral buffered formalin for 1 week at room temperature before the fixed lungs were transferred to 70% ethanol. Histopathology was carried out on two animals from each of the following groups: PBS only+no challenge, placebo+beta challenge, placebo+delta challenge, bivalent high Al(OH)3 and CPG 1018+beta challenge, Bivalent high Al(OH)3 and CPG 1018+delta challenge. Animals from the Bivalent high Al(OH)3 group did not undergo histopathology and immunohistochemistry. Samples were processed by WaxIt (Vancouver, B.C., Canada) by standard paraffin embedding methods and blocks were cut into 5 μm thick sections, placed on glass slides, and subjected to hematoxylin and Eosin (H&E).

Immunohistochemical (IHC) staining was completed at the NRC modified protocol F on the Bond-Max III fully automated staining system (Leica Biosystems, Wetzlar, Germany) was employed. All reagents from the Bond Polymer Refine Detection Kit were used. SARS-CoV-2 was detected using mouse anti-SARS-CoV-2 nucleocapsid monoclonal antibody (1:5000, R&D System, Minneapolis, MN, USA). Following deparaffinization and rehydration, sections were pre-treated with the Epitope Retrieval Solution 1 (ER1, Citrate buffer, pH 5.0) or Epitope Retrieval Solution 2 (ER2, EDTA buffer, pH 8.8) at 98° C. for 20 min. After washes, non-specific endogenous peroxidases were quenched using peroxidase block for 5 min, followed by another washed, and then incubated for 15 min at room temperature with primary antibodies. A mouse-on-mouse superblock was applied for 15 min prior to addition of anti-SARS-CoV-2 nucleocapsid antibody (PowerVision IHC/ISH Super Blocking, Leica Biosystems, Wetzlar, Germany). After addition of primary and subsequent washes, sections were incubated with polymer refine for 8 min at room temperature and developed with 3, 3′-diaminobenzidine (DAB) chromogen for 10 min. Sections were then washed and counterstained for 6 min with hematoxylin, dehydrated, cleared and mounted. Negative controls included omission of primary antibody and incubation with secondary antibody alone as well as lung tissue from naïve animals. IHC slides were scanned at 20× magnification using a Zeiss Axio Scan. Z1 digital slide scanner capable of brightfield imaging.

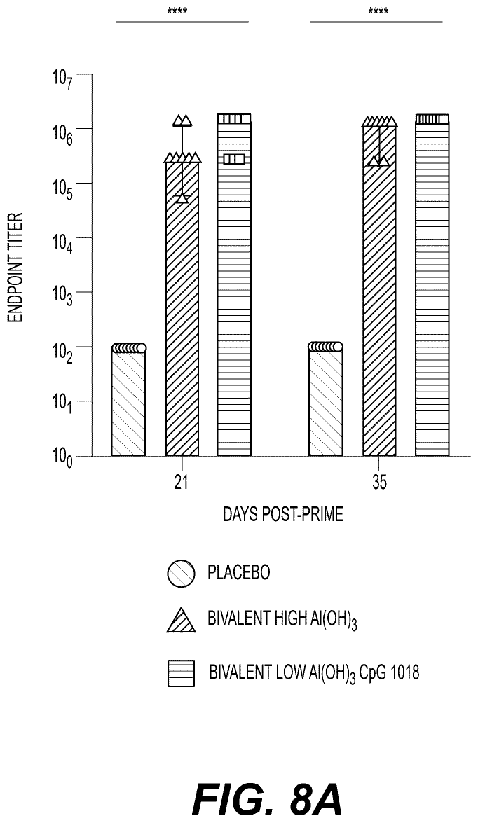

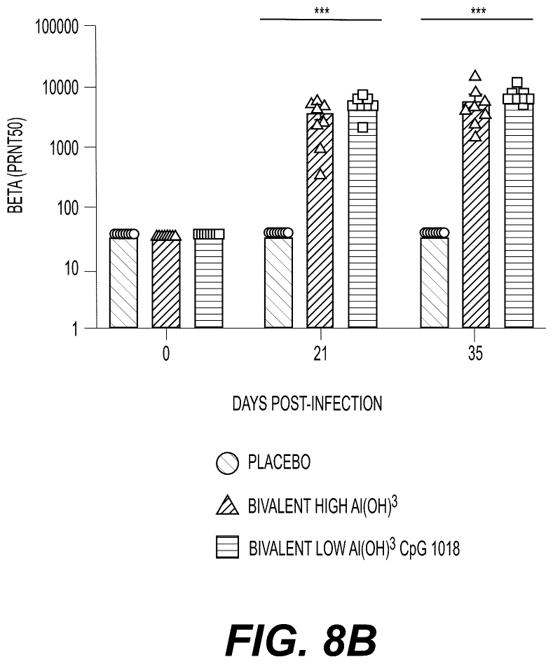

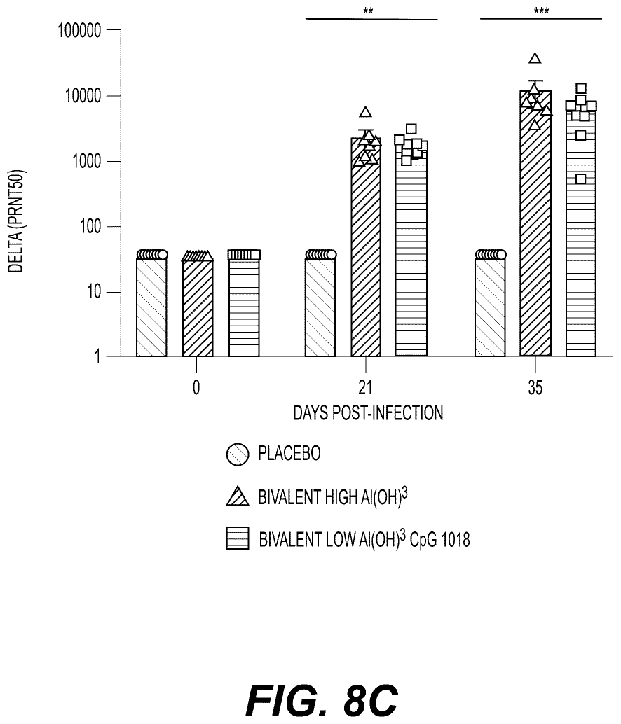

All tests were performed as discrete tests, with samples from each animals tested independently. All statistical analysis was performed using GraphPad Prism version 10.0.0 for Windows (GraphPad Prism Software, Massachusetts, USA, www.graphpad.com). For the mouse study, Mann-Whitney t-tests were performed to compare immune responses between each group. For serological and viral load comparisons non-parametric one-way ANOVA with multiple comparisons to placebo were performed. Gaussian distribution was not assumed. (Kruskal-Wallis test with Dunn's multiple comparisons test). All data are presented as Geometric Mean±95% Confidence Interval (CI) unless otherwise stated. Statistical significance is denoted by asterisks in the figures (*P<0.05, **P<0.01, ***P<0.001, and ****P<0.0001), where alpha=0.05 (FIGS. 8A-8C).

Other embodiments and uses of the invention will be apparent to those skilled in the art from consideration of the specification and practice of the invention disclosed herein. All references cited herein, including all publications, and all U.S. and foreign patents and patent applications are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A method for the manufacture of an immunogenic composition comprising:
providing an RNA sequence that encodes an antigenic determinant;
expressing an antigen from the RNA sequence in a cell line; and
collecting the antigen expressed.

2. The method of claim 1, wherein the antigenic determinant is an immunologically active antigen.

3. The method of claim 1, wherein the immunologically active antigen is derived from a species, subspecies or serotypes of Staphylococcus, Streptococcus, Salmonella, Pneumococcus, Campylobacter, Pseudomonas, Neisseria, Mycobacteria, Corynebacteria, Enterobacter, Vibrio, Bordetella, Clostridium, Yersinia, Listeria, Treponema, or another infectious microorganism.

4. The method of claim 1, further comprising combining the antigen to a biological substance to form an immunogenic composition.

5. The method of claim 4, wherein the biological substance comprises a carrier protein.

6. The method of claim 5, wherein the carrier protein is selected from the group consisting of tetanus toxoid, diphtheria toxoid, CRM197, tetanus toxoid fragments (TTHc), N. meningitidis protein PorB, RSV virus proteins, B. Pertussis proteins, Pertussis toxoid (PT), adenylate cyclase toxin (ACT), 69 KDa protein, Human Papilloma viral protein antigens, Human Papilloma virus VLP forms, Hepatitis B virus core antigen, Hepatitis B virus VLP forms, derivatives of HBsAg, and/or combinations thereof.

7. The method of claim 1, further comprising an adjuvant.

8. The method of claim 7, wherein the adjuvant comprises aluminum salt, calcium phosphate, a liposome of monophosphoryl lipid A (MPLA), saponin QS-21, TLR ligands, and/or a potent TLR4/7/8/9 agonists.

9. The method of claim 4, wherein the carrier protein is conjugated to the antigen.

10. The method of claim 4, wherein the carrier protein is a toll-like receptor agonist.

11. An antigen manufactured by the method of claim 1.

12. A method of administering the antigen of claim1 to treat or prevent an infection.
